(19) United States
(12) Patent Application Publication
(10) Pub. No.: US 2022/0072174 A1
(43) Pub. Date: Mar. 10, 2022

(54) System and Method for Sterilizing Cash Exchanges

(71) Applicant: Mark Pragano, Naugatuck, CT (US)

(72) Inventor: Mark Pragano, Naugatuck, CT (US)

(21) Appl. No.: 17/471,133

(22) Filed: Sep. 9, 2021

Related U.S. Application Data
(60) Provisional application No. 63/076,273, filed on Sep. 9, 2020.

(51) Int. Cl. A61L 2/24; A61L 2/10; A61L 2/18

(52) U.S. Cl. CPC A61L 2/24; A61L 2/10; A61L 2202/14; A61L 2202/122; A61L 2/18

(57) ABSTRACT

A system and method sterilizes cash exchanges. The system includes a sanitization system and a transaction station. The transaction station may any type of transaction station such as, but not limited to, a teller window, a vacuum air tube (VAT) station, or an automated teller machine (ATM). The transaction station includes a user access area. The user access area is the main access point for the user when executing a transaction. For example, the user access area can be a transaction drawer, a tubular container and a container-receiving pocket, or a user ATM interface. The sanitization system includes an ultraviolet light (UVL) device which is the primary means to sterilize a cash exchanged being executed by the user. The sanitization system also includes a covering mechanism which is used to enclose the user access area in order to prevent a user from being exposed to ultraviolet (UV) lighting.

The current application claims a priority to the U.S. Provisional Patent application Ser. No. 63/076,273 filed on Sep. 9, 2020.

### FIELD OF THE INVENTION

The present invention generally relates to sanitizing/disinfecting systems and methods. More specifically, the present invention provides a system and method for sterilizing cash exchanges.

### BACKGROUND OF THE INVENTION

The present invention provides a system and method for sterilizing bank exchanges. The present invention, also known as the Prag Germ Safe Banking Transaction, utilizes Ultraviolet Germicidal Irradiation (UVGI) to create germ-free transactions including, but not limited to, bank teller transactions through cash drawers, drive-up window drawers, automated teller machines (ATMs), or teller cash drawers. The Prag Germ Safe Transaction includes any process or apparatus that utilizes ultraviolet (UV) lighting to sanitize and/or disinfect the items exchanged during a financial transaction, such as money or deposit slips, as well as the surface or objects used during the financial transaction, such as pin pads, ATMs, cash counters, etc. The UV light radiated is preferably on the ultraviolet-c (UVC) wavelength which is known to work as germicidal method. The present invention can further incorporate other sanitizing/disinfecting systems or methods.

The present invention can be installed internally or externally on the appropriate structure to clean monetary or related items going in and out of an ATM machines, cash counters, etc. As items are deposited in, the UV lighting will disinfect the different items being exchanged as well as the internal/external components. A covering mechanism can be provided to cover keypads the various external components public after each usage. When the covering mechanism is engaged, the present invention will disinfect the different external components such as the touchpad, the screen, etc. The UV lighting can also be installed internally so that cash being sent out of machine will be exposed to UV lighting to disinfect the bills leaving the machine. Additional benefits and features are further discussed in the sections below.

### BRIEF DESCRIPTION OF THE DRAWINGS

FIG. 1 is a diagram illustrating the overall system of the present invention.

FIG. 2 is as flowchart illustrating the overall process for the method of the present invention.

FIG. 3 is a flowchart illustrating the subprocess of using a sensor to detection motion away from the transaction station.

FIG. 4 is a flowchart illustrating the subprocess of further sterilizing the user access area.

FIG. 5 is a diagram displaying the transaction station and sanitization system in an automated teller machine (ATM) embodiment.

FIG. 6 is a diagram displaying the drop-down screen in the process of unraveling to cover the user ATM interface.

FIG. 7 is a diagram displaying the drown-down screen fully unraveled to cover the user ATM interface.

FIG. 8 is a flowchart illustrating the subprocess on how the sanitization system is used for an ATM.

FIG. 9 is a diagram displaying the transaction station in a vacuum air tube (VAT) station embodiment.

FIG. 10 is a diagram displaying the tubular container and the plurality of slots.

FIG. 11 is a flowchart illustrating the subprocess on how the sanitization system is used for a VAT station.

FIG. 12 is a diagram displaying the transaction station in a teller window embodiment.

FIG. 13 is a diagram displaying the through-wall transaction drawer and the drawer lid in an open position.

FIG. 14 is a diagram displaying the through-wall transaction drawer and the drawer lid in a closed position.

FIG. 15 is a flowchart illustrating the subprocess on how the sanitization system is used for a teller window.

### DETAIL DESCRIPTIONS OF THE INVENTION

All illustrations of the drawings are for the purpose of describing selected versions of the present invention and are not intended to limit the scope of the present invention.

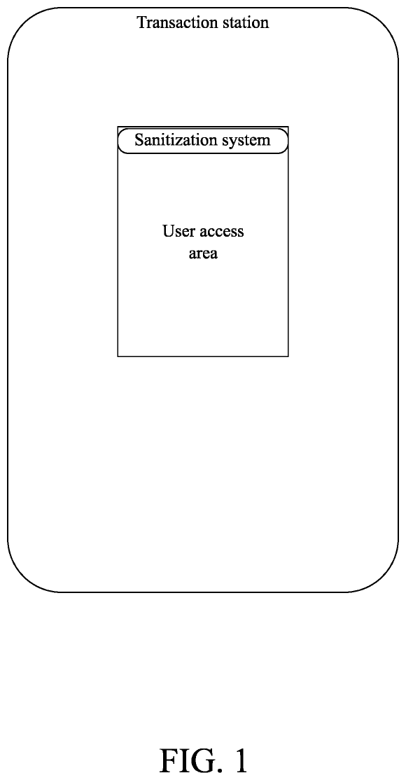

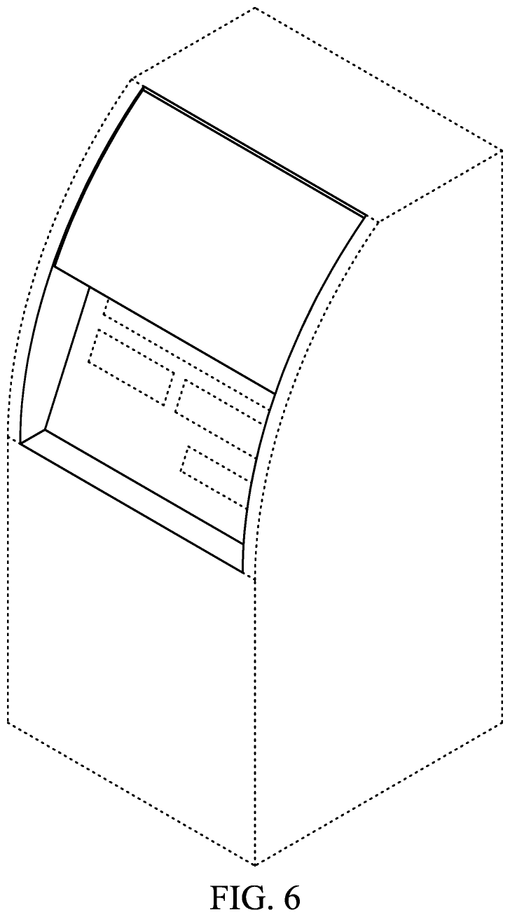

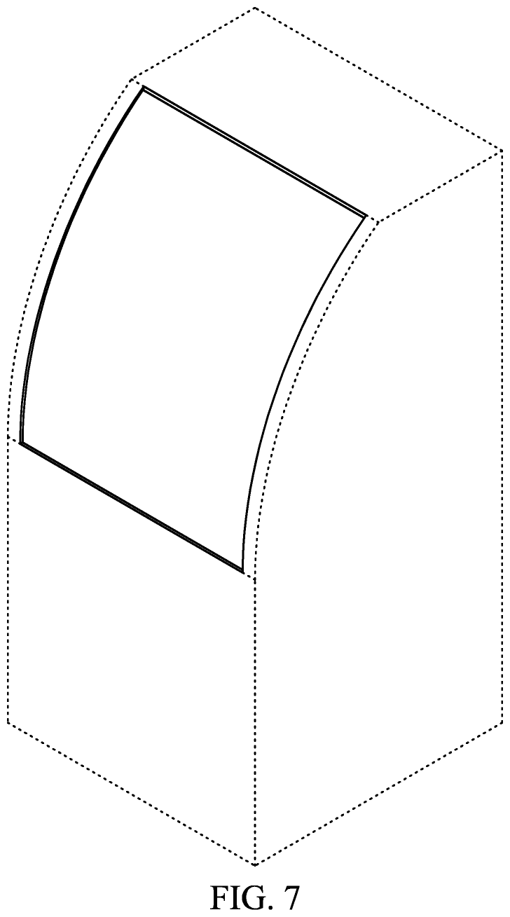

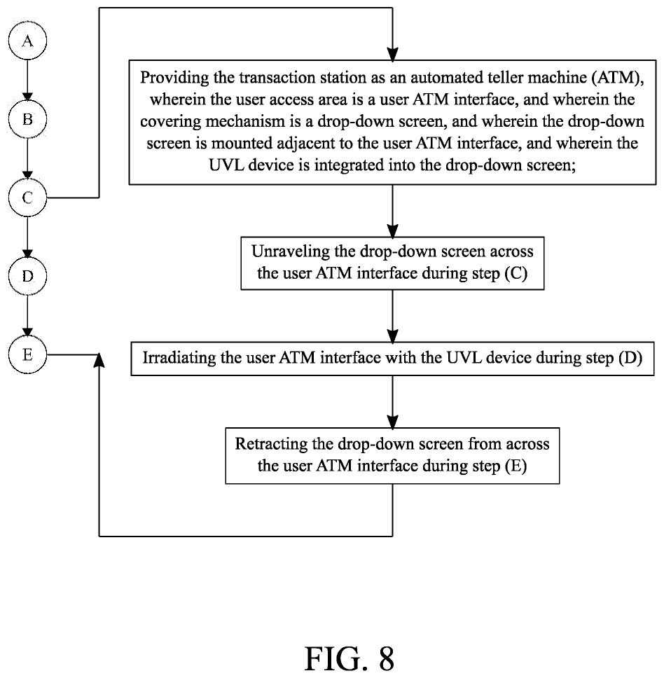

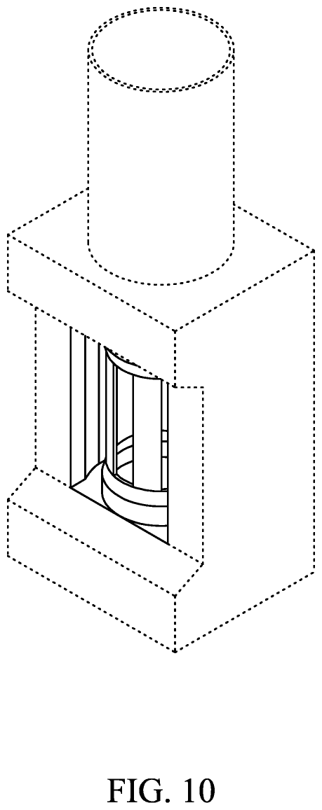

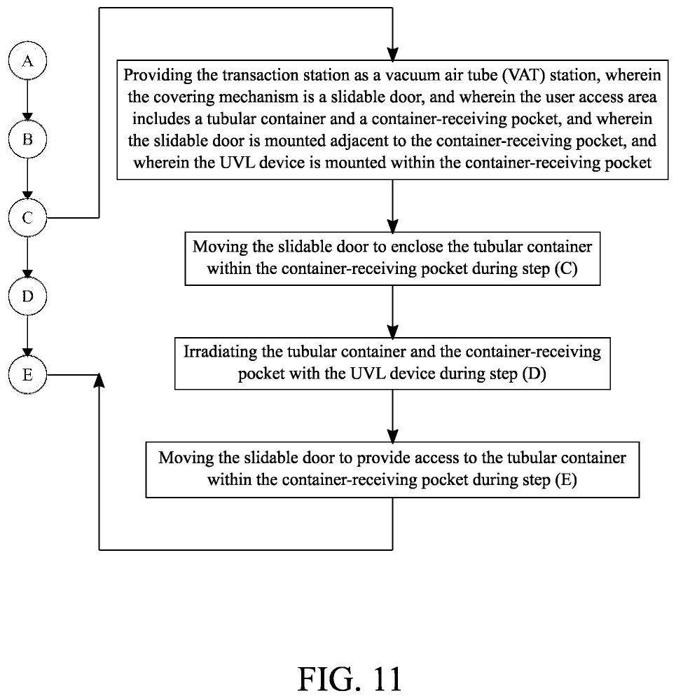

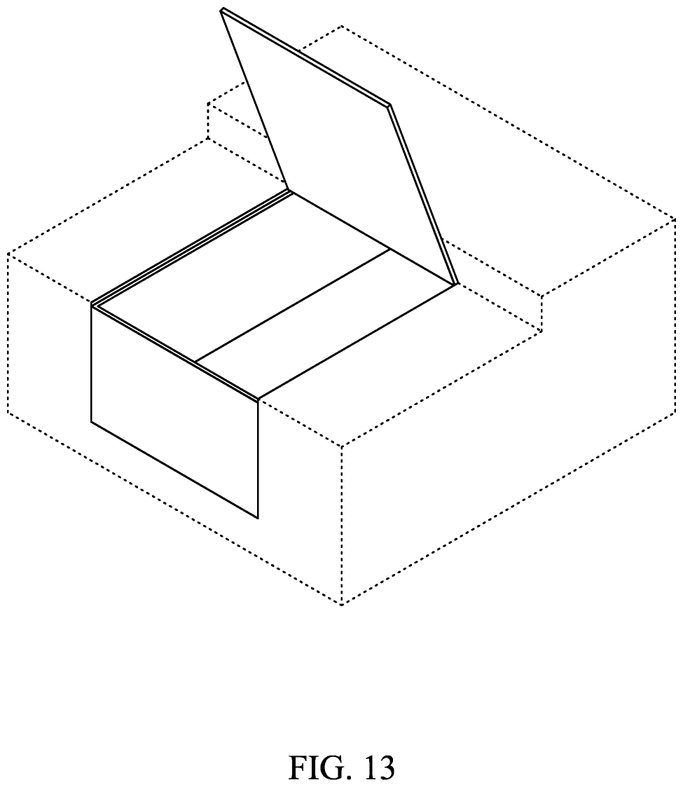

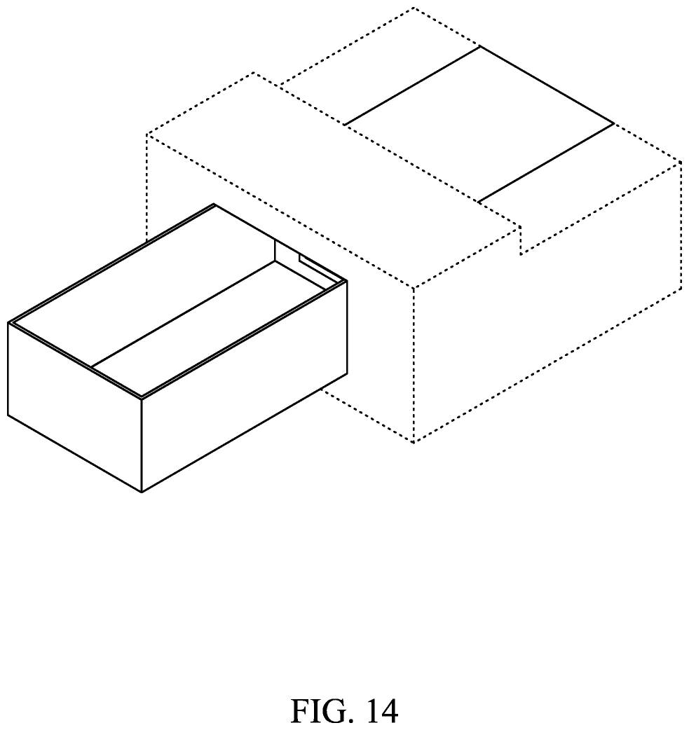

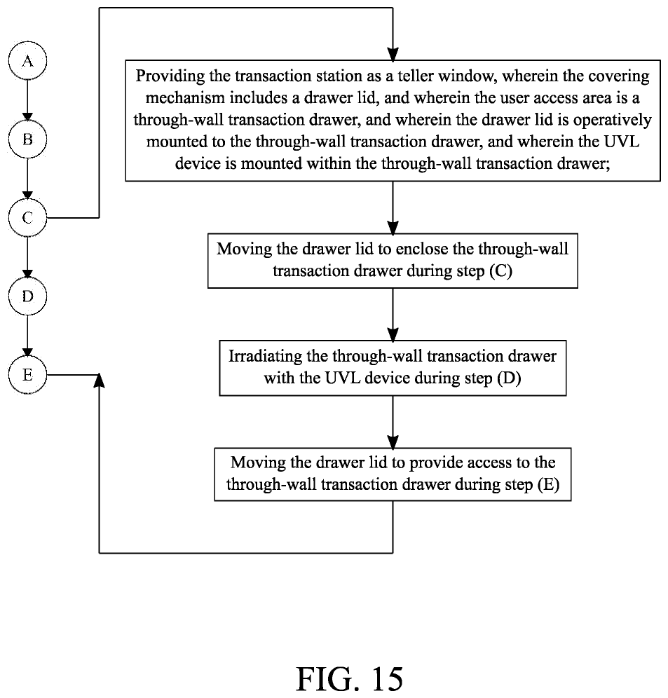

In reference to FIGS. 1 through 15, the present invention is a system and method for sterilizing cash exchanges. With reference to FIG. 1, the system of the present invention includes a sanitization system and a transaction station (Step A). The transaction station may cover any type of transaction station such as, but not limited to, a teller window, a vacuum air tube (VAT) station, or an automated teller machine (ATM). The transaction station includes a user access area. The user access area is the main access point for the user when executing a transaction. For example, the user access area can be a transaction drawer of a teller window, the tubular container and container-receiving pocket of a VAT station, or the user ATM interface of an ATM. The sanitization system includes an ultraviolet light (UVL) device which is the primary means to sterilize a cash exchanged being executed by the user. The sanitization system also includes a covering mechanism which is used to enclose the user access area in order to ensure that only the user access area is exposed to ultraviolet (UV) lighting. Further, the sanitization system is communicably coupled to the transaction station in order to ensure sterilization process is effectively executed. In more detail, the sanitization system may be directly coupled to the transaction station through a controller or wirelessly coupled to the transaction station through wireless communication modules.

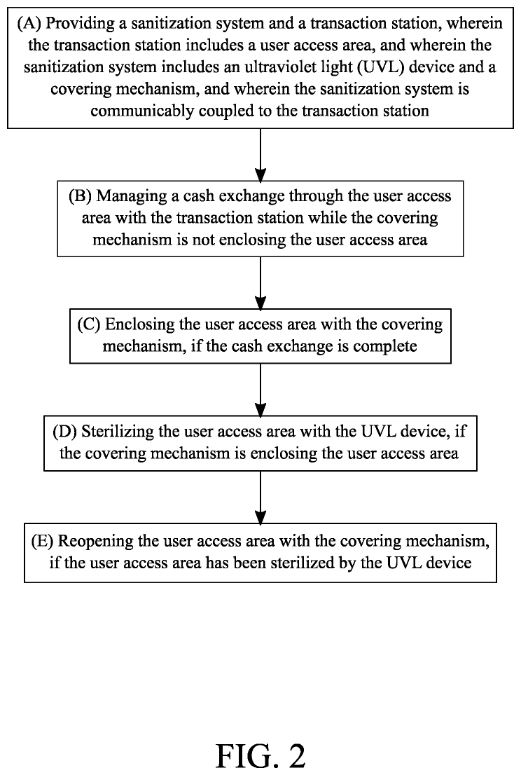

With reference to FIG. 2, the method of the present invention follows an overall process for sterilizing cash exchanges. The transaction station manages a cash exchange through the user access while the covering mechanism is not enclosing the user access area (Step B). The cash exchange may be any type of cash exchange such as, but not limited to, depositing cash, or withdrawing cash. Further, the covering mechanism should not be enclosing the user access area in order for the user to insert his or her bank card, input credentials, deposit cash, and/or withdraw cash. After the cash exchange is complete, the covering mechanism encloses the user access area (Step C). Step C is executed in order to prevent the user from exposed to UV lighting once sterilization is executed. The UVL device then sterilizes the user access area if the covering mechanism is enclosing the user access area (Step D). The enclosure of the user access area also ensures that any items and/or any objects touched within or part of the user access area are fully sterilized for another cash exchange. Finally, the covering mechanism reopens the user access area if the user access has been sterilized by the UVL device (Step E). In more detail, another user can safely perform a cash exchange through the present invention due to the user access area being sterilized after each cash exchange.

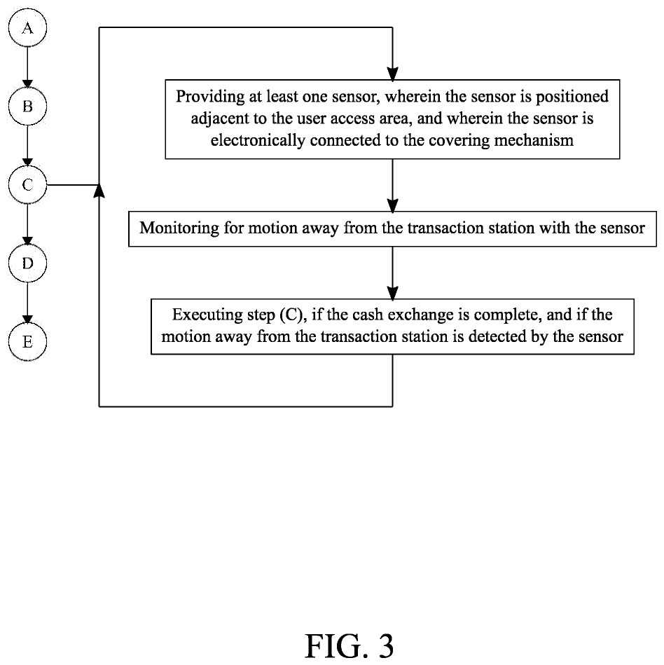

In order to safely operate the UVL device without a user being exposed to UV lighting and with reference to FIG. 3, the following subprocess is executed. The system of the present invention further includes at least one sensor. The sensor is positioned adjacent to the user access area and the sensor is electronically connected to the covering mechanism. The at least one sensor includes more than one sensor that detects motion. Thus, the sensor monitors for motion away from the transaction station. In more detail, the sensor is used to detect when there is no motion around the transaction station. Step C is executed if the cash exchange is complete, and if the motion away form the transaction station is detected by the sensor. Thus, the present invention safely operates the UVL device without a user being exposed to UV lighting.

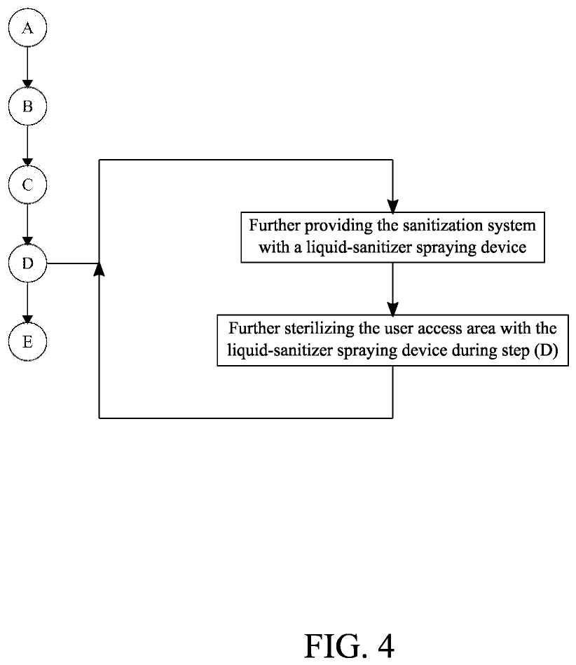

In order to further sterilize the user access area and with reference to FIG. 4, the following subprocess is executed. The sanitization system is further provided with a liquid-sanitizer spraying device. In more detail, the liquid-sanitizer spraying device includes a spray mechanism that can output a liquid disinfectant. The liquid-sanitizer spraying device further sterilizes the user access area during Step D. In more detail, the liquid-sanitizer spraying device is used to eliminate any bacteria that may remain after the UVL device is used.

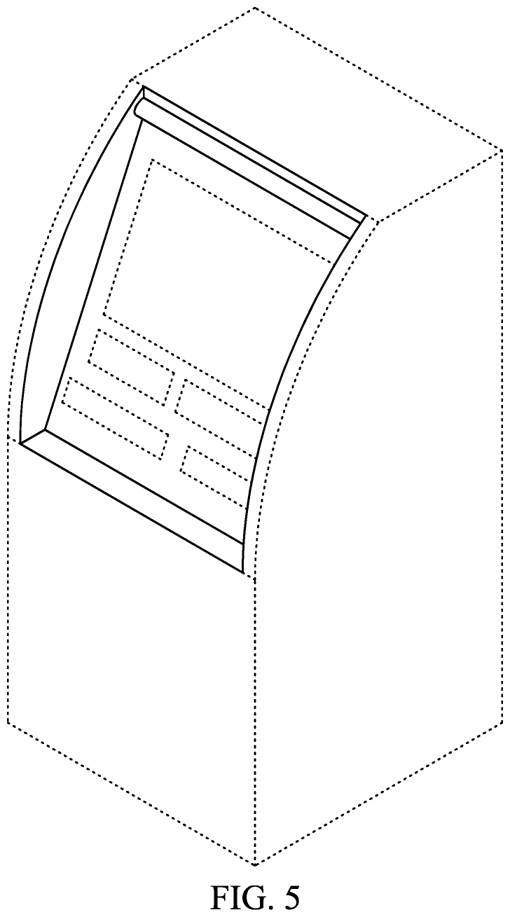

In an embodiment where the transaction station is provided as an ATM and with reference to FIGS. 5 through 8, the following subprocess is executed. The user access area is a user ATM interface. In more detail, the user ATM interface is the interface that includes buttons, a display which may be a touch-screen display, inputs for cash, checks, and a bank card, and outputs for cash and receipts. The covering mechanism is a drop-down screen which mounted adjacent to the user ATM interface in order to fully cover the user ATM interface when the covering mechanism is in the closed state. The UVL device is integrated into the drop-down screen. In more detail, the UVL device is a UVL strip that is mounted within the roll tube of the drop-down screen. This adds more safety for a user to not be exposed to UV lighting in which the UVL device is only exposed when the screen is unraveled. The drop-down screen is unraveled across the user ATM interface during Step C in order to prevent a user from being exposed to UV lighting. The UVL device then irradiates the user ATM interface during Step D. In more detail, the UVL device irradiates the buttons, the display, the inputs, and the outputs of the user ATM interface for 5 to 7 seconds in order to sterilize the user ATM interface. After the user ATM interface has been sterilized, the drop-down screen is retracted from across the user ATM interface during Step E in order to allow another user to perform a cash exchange.

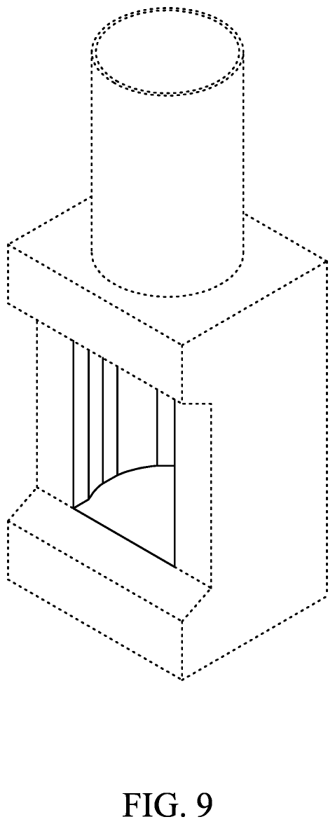

In an embodiment where the transaction station is provided as a VAT station and with reference to FIGS. 9 through 11, the following subprocess is executed. The covering mechanism is a slidable door and the user access area includes a tubular container and a container-receiving pocket. The slidable door is mounted adjacent to the container-receiving pocket in order to close-off or allow access to the inside of the container-receiving pocket. The container-receiving pocket is an area that houses the tubular container which is used to hold cash and other required bank items. The UVL device is mounted within the container-receiving pocket. In more detail, the UVL device is one or more UV lighting strips that are mounted within the container-receiving pocket in order to emit UV lighting onto the tubular container and the inner surface area of the container-receiving pocket. The slidable door is moved to enclose the tubular container within the container-receiving pocket during Step C. In more detail, the slidable door can be automatic in which the slidable door selectively slides open or closed with a push of a button or a user can manually slide the slidable open or closed through a handle of the slidable door. The UVL device then irradiates the tubular container and the container-receiving pocket during Step D. In more detail, the UVL device irradiates the tubular container and the container-receiving pocket for 3 to 5 seconds in order to sterilize the tubular container and the container-receiving pocket. Moreover, in order to sterilize the contents within the tubular container which may be cash and other required bank items, the tubular container includes a plurality of slots. Each of the plurality of slots laterally traverse into the tubular container in order to partially reveal the contents within the tubular container. Further, the plurality of slots is distributed about the tubular container and the plurality of slots is configured to allow electromagnetic radiation to evenly enter the tubular container. Thus, the contents within the tubular container are also sterilized during Step D. Finally, the slidable door is moved to provide access to the tubular container within the container-receiving pocket during Step E. Thus, the user can retrieve any contents from the tubular container if necessary or another user can perform a cash exchange.

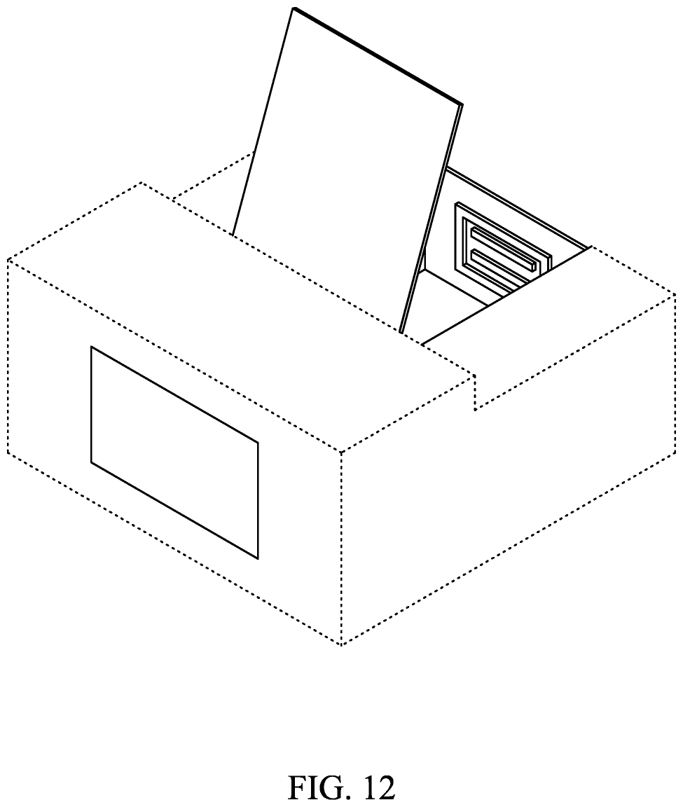

In an embodiment where the transaction station is provided as a teller window and with reference to FIGS. 12 through 15, the following subprocess is executed. The covering mechanism is a drawer lid and the user access area includes a through-wall transaction drawer. In more detail, the through-wall transaction drawer is a drawer that can extend from the teller window for users and retracts into the teller window for bank tellers. The drawer lid is operatively mounted o the through-wall transaction drawer in order to close-off or allow access to the inside of the through-wall transaction drawer. In more detail, the drawer lid is hingedly mounted against a wall of the teller window in which the drawer lid is primarily operated when the through-wall transaction drawer is extended for the user. Further, the UVL device is mounted within the through-wall transaction drawer. In more detail, the UVL device is one or more UV lighting strips that are mounted within the through-wall transaction drawer in order to emit UV lighting onto any items such as cash and other required bank items that are placed inside the through-wall transaction drawer and the inner surface area of the through-wall transaction drawer. The drawer lid is moved to enclose the through-wall transaction drawer during Step C. In more detail, the drawer lid can be automatic in which the drawer lid selectively opens or closes with a push of a button or a user can manually open or close the drawer lid through a handle of the drawer lid. The UVL device then irradiates the through-wall transaction drawer during Step D. In more detail, the UVL device irradiates the contents within the through-wall transaction drawer and the inner surface area of the through-wall transaction drawer for 3 to 5 seconds in order to sterilize the through-wall transaction drawer. Finally, the drawer lid is moved to provide access to the through-wall transaction drawer during Step E. Thus, the user can retrieve any contents from the through-wall transaction drawer if necessary or another user can perform a cash exchange.

Although the invention has been explained in relation to its preferred embodiment, it is to be understood that many other possible modifications and variations can be made without departing from the spirit and scope of the invention as hereinafter claimed.

What is claimed is:

1. A method for sterilizing cash exchanges, the method comprising the steps of:
(A) providing a sanitization system and a transaction station, wherein the transaction station includes a user access area, and wherein the sanitization system includes an ultraviolet light (UVL) device and a covering mechanism, and wherein the sanitization system is communicably coupled to the transaction station;
(B) managing a cash exchange through the user access area with the transaction station while the covering mechanism is not enclosing the user access area;
(C) enclosing the user access area with the covering mechanism, if the cash exchange is complete;
(D) sterilizing the user access area with the UVL device, if the covering mechanism is enclosing the user access area; and
(E) reopening the user access area with the covering mechanism, if the user access area has been sterilized by the UVL device.

2. The method for providing germ-free bank transactions, the method as claimed in claim 1 comprising the steps of:
providing at least one sensor, wherein the sensor is positioned adjacent to the user access area, and wherein the sensor is electronically connected to the covering mechanism;
monitoring for motion away from the transaction station with the sensor; and
executing step (C), if the cash exchange is complete, and if the motion away from the transaction station is detected by the sensor.

3. The method for providing germ-free bank transactions, the method as claimed in claim 1 comprising the steps of:
further providing the sanitization system with a liquid-sanitizer spraying device; and
further sterilizing the user access area with the liquid-sanitizer spraying device during step (D).

4. The method for method for sterilizing exchanges of cash, the method as claimed in claim 1 comprising the steps of:
providing the transaction station as an automated teller machine (ATM), wherein the user access area is a user ATM interface, and wherein the covering mechanism is a drop-down screen, and wherein the drop-down screen is mounted adjacent to the user ATM interface, and wherein the UVL device is integrated into the drop-down screen;
unraveling the drop-down screen across the user ATM interface during step (C);
irradiating the user ATM interface with the UVL device during step (D); and
retracting the drop-down screen from across the user ATM interface during step (E).

5. The method for method for sterilizing exchanges of cash, the method as claimed in claim 4, wherein the UVL device irradiates the user ATM interface for 5 to 7 seconds.

6. The method for providing germ-free bank transactions, the method as claimed in claim 1 comprising the steps of:
providing the transaction station as a vacuum air tube (VAT) station, wherein the covering mechanism is a slidable door, and wherein the user access area includes a tubular container and a container-receiving pocket, and wherein the slidable door is mounted adjacent to the container-receiving pocket, and wherein the UVL device is mounted within the container-receiving pocket;
moving the slidable door to enclose the tubular container within the container-receiving pocket during step (C);
irradiating the tubular container and the container-receiving pocket with the UVL device during step (D); and
moving the slidable door to provide access to the tubular container within the container-receiving pocket during step (E).

7. The method for method for sterilizing exchanges of cash, the method as claimed in claim 6, wherein the UVL device irradiates the tubular container and the container-receiving pocket for 3 to 5 seconds.

8. The method for method for sterilizing exchanges of cash, the method as claimed in claim 6, wherein the tubular container includes a plurality of slots, and each of the plurality of slots laterally traverses into the tubular container, and wherein the plurality of slots is distributed about the tubular container, and wherein the plurality of slots is configured to allow electromagnetic radiation to evenly enter the tubular container.

9. The method for providing germ-free bank transactions, the method as claimed in claim 1 comprising the steps of:
providing the transaction station as a teller window, wherein the covering mechanism includes a drawer lid, and wherein the user access area is a through-wall transaction drawer, and wherein the drawer lid is operatively mounted to the through-wall transaction drawer, and wherein the UVL device is mounted within the through-wall transaction drawer;
moving the drawer lid to enclose the through-wall transaction drawer during step (C);
irradiating the through-wall transaction drawer with the UVL device during step (D); and
moving the drawer lid to provide access to the through-wall transaction drawer during step (E).

10. The method for method for sterilizing exchanges of cash, the method as claimed in claim 9, wherein the UVL device the through-wall transaction drawer for 3 to 5 seconds.
